(19) United States
(12) Patent Application Publication
(10) Pub. No.: US 2025/0107710 A1
(43) Pub. Date: Apr. 3, 2025

(54) MEDICAL INFORMATION SYSTEM, MODALITY APPARATUS, INFORMATION PROCESSING METHOD, AND PROGRAM

(71) Applicant: FUJIFILM Corporation, Tokyo (JP)

(72) Inventor: Wataru YOKOHAMA, Chiba (JP)

(73) Assignee: FUJIFILM Corporation, Tokyo (JP)

(21) Appl. No.: 18/898,618

(22) Filed: Sep. 26, 2024

(30) Foreign Application Priority Data
Sep. 29, 2023 (JP) 2023-170722

(51) Int. Cl. A61B 5/00

(52) U.S. Cl. CPC A61B 5/0033; A61B 5/7435

(57) ABSTRACT

Provided are a medical information system, a modality apparatus, an information processing method, and a program capable of confirming and selecting an appropriate imaging protocol corresponding to an examination instruction of an examination order. A medical information system includes: an information system that generates an examination order including an examination instruction for an image diagnosis examination; and a modality apparatus that captures a medical image of a subject in accordance with the examination order, in which the modality apparatus includes a storage device that pre-associates and stores the examination instruction and one or more imaging protocols, and one or more processors that execute processing of acquiring the examination order from the information system and displaying the examination instruction of the acquired examination order and the one or more imaging protocols pre-associated with the examination instruction.

### CROSS-REFERENCE TO RELATED APPLICATION

The present application claims priority under 35 U.S.C § 119(a) to Japanese Patent Application No. 2023-170722 filed on Sep. 29, 2023, which is hereby expressly incorporated by reference, in its entirety, into the present application.

### BACKGROUND OF THE INVENTION

### 1. Field of the Invention

The present disclosure relates to a medical information system, a modality apparatus, an information processing method, and a program.

### 2. Description of the Related Art

JP2020-187542A describes an imaging support apparatus comprising: a determination unit that determines, based on contents of patient information regarding a patient, order information for requesting imaging of the patient, and instruction information related to performing the imaging, which is created according to the order information, an imaging condition in a case in which the imaging of the patient is performed with a modality, in a form of an imaging protocol that can be read by the modality; and an output unit that outputs the imaging protocol determined by the determination unit to the modality.

In the technology described in JP2020-187542A, by performing machine learning using a set of change factors of patient information, an examination order, and an examination instruction, which are acquired in the past, as learning data, and an imaging protocol used in the learning data, as training data, a trained model that infers the imaging protocol from the set of change factors of the patient information, the examination order, and the examination instruction is created. Then, during operation, the set of change factors is input to the trained model on an image diagnosis division information management system side, the imaging protocol is obtained as an output, and the imaging protocol is transmitted to the modality.

### SUMMARY OF THE INVENTION

The operation of creating the examination order is a significant workload for busy physicians, such as those involved in image interpretation, and there is a need for order creation to be accurate and less burdensome. Meanwhile, an examination staff such as a radiologist who operates a modality apparatus used in an image diagnosis examination carefully confirms the examination order of the physician such that an incorrect imaging protocol due to a human error is not selected, and this imposes a significant burden. Further, with the advancement of imaging technology in recent years, there has been a diversification of examination contents, leading to an increase and complexity of imaging protocols. An examination staff with little knowledge and experience may sometimes be unsure of which imaging protocol to select. In such a case, the examination staff with little knowledge and experience can directly consult experienced technicians or physicians. However, both parties may be busy and unable to spare time immediately, which may lead to a case in which the confirmation takes some time. As a result, there is a problem such as delays in starting the examination.

According to the technology described in JP2020-187542A, the burden on a side of the physician who issues the examination instruction is reduced in a radiology information system (RIS). However, since the examination staff needs to confirm that the received imaging protocol is appropriate, and the content of the imaging protocol may be updated or changed as appropriate, it is not sufficient to reduce the burden on the examination staff and prevent errors.

The present disclosure has been made in view of such circumstances, and an object of the present disclosure is to provide a medical information system, a modality apparatus, an information processing method, and a program capable of confirming and selecting an appropriate imaging protocol corresponding to an examination instruction of an examination order.

According to a first aspect of the present disclosure, there is provided a medical information system including: an information system that generates an examination order including an examination instruction for an image diagnosis examination; and a modality apparatus that captures a medical image of a subject in accordance with the examination order, in which the modality apparatus includes a storage device that pre-associates and stores the examination instruction and one or more imaging protocols, and one or more processors that execute processing of acquiring the examination order from the information system and displaying the examination instruction of the acquired examination order and the one or more imaging protocols pre-associated with the examination instruction.

According to the first aspect, the examination instruction and the one or more imaging protocols are pre-associated and stored in the storage device of the modality apparatus. In the modality apparatus that acquires the examination order from the information system, the examination instruction designated in the examination order and the one or more imaging protocols pre-associated with the examination instruction can be displayed. As a result, an operator such as an examination staff can confirm and select an appropriate imaging protocol corresponding to the examination instruction of the examination order.

According to a second aspect, in the medical information system according to the first aspect, the one or more processors may be configured to execute processing of displaying a setting screen for accepting a setting operation to associate the one or more imaging protocols corresponding to the examination instruction.

According to a third aspect, in the medical information system according to the first or second aspect, the one or more processors may be configured to execute processing for provisional registration of storing identification information determined for each type of the examination instruction and a plurality of imaging protocols related to an examination site according to the examination instruction in association with each other in the storage device.

According to a fourth aspect, in the medical information system according to the third aspect, the one or more processors may be configured to display the plurality of imaging protocols associated with the identification information and display a screen for prompting an operator to select any of the imaging protocols, in a case in which the provisional registration has been made for the examination instruction of the examination order acquired from the information system.

According to a fifth aspect, in the medical information system according to any one of the first to fourth aspects, the one or more processors may be configured to execute processing for final registration of storing identification information determined for each type of the examination instruction and one imaging protocol in association with each other in the storage device.

According to a sixth aspect, in the medical information system according to the fifth aspect, the one or more processors may be configured to display the imaging protocol uniquely associated with the identification information and execute automatic selection of the uniquely associated imaging protocol, in a case in which the final registration has been made for the examination instruction of the examination order acquired from the information system.

According to a seventh aspect, in the medical information system according to any one of the first to sixth aspects, the examination order may include coordination necessity information indicating a necessity of coordination between the information system and the modality apparatus.

According to an eighth aspect, in the medical information system according to the seventh aspect, the one or more processors may be configured to control whether or not to display the pre-associated one or more imaging protocols according to the coordination necessity information included in the examination order acquired from the information system.

According to a ninth aspect, in the medical information system according to any one of the first to eighth aspects, the examination order may include identification information determined for the examination instruction.

According to a tenth aspect, in the medical information system according to any one of the first to ninth aspects, a common symbol that is unchangeable between the information system and the modality apparatus may be assigned to a pair of the pre-associated examination instruction and imaging protocol.

According to an eleventh aspect, there is provided a modality apparatus that captures a medical image of a subject in accordance with an examination order including an examination instruction for an image diagnosis examination, the modality apparatus including: a storage device that pre-associates and stores the examination instruction and one or more imaging protocols; and one or more processors that execute processing of acquiring the examination order and displaying the examination instruction of the acquired examination order and the one or more imaging protocols pre-associated with the examination instruction.

In the modality apparatus according to the eleventh aspect, the same specific aspect as that of the medical information system according to the second to tenth aspects can be included.

According to a twelfth aspect, there is provided an information processing method of coordinating an information system that generates an examination order including an examination instruction for an image diagnosis examination with a modality apparatus that captures a medical image of a subject in accordance with the examination order, the information processing method including: causing one or more processors of the modality apparatus to pre-associate and store the examination instruction and one or more imaging protocols in a storage device; and causing the one or more processors to execute processing of acquiring the examination order from the information system and of displaying the examination instruction of the acquired examination order and the one or more imaging protocols pre-associated with the examination instruction.

In the information processing method according to the twelfth aspect, the same specific aspect as that of the medical information system according to the second to tenth aspects can be included.

According to a thirteenth aspect, there is provided a program for causing a computer to implement a function of coordinating an information system that generates an examination order including an examination instruction for an image diagnosis examination with a modality apparatus that captures a medical image of a subject in accordance with the examination order, the program causing the computer to implement: a function of pre-associating and storing the examination instruction and one or more imaging protocols in a storage device; and a function of acquiring the examination order from the information system and displaying the examination instruction of the acquired examination order and the one or more imaging protocols pre-associated with the examination instruction.

In the program according to the thirteenth aspect, the same specific aspect as that of the medical information system according to the second to tenth aspects can be included.

The present disclosure also includes a non-transitory computer-readable recording medium (computer-readable medium), which is a tangible object such as an optical disk, a semiconductor memory, and a magnetic disk, on which the program according to the thirteenth aspect is recorded.

According to the present disclosure, it is possible to confirm and select an appropriate imaging protocol corresponding to the examination instruction of the examination order. According to the present disclosure, the burden on the examination staff or the like who handles the modality apparatus is reduced, thereby enabling suppression of selection errors in imaging protocols and enhancing the efficiency of examination operations.

### BRIEF DESCRIPTION OF THE DRAWINGS

FIG. 1 is a block diagram schematically showing an exemplary configuration of a medical information system.

FIG. 2 is a perspective view schematically showing a configuration of a magnetic resonance imaging (MRI) apparatus that is an example of a modality apparatus.

FIG. 3 is a block diagram showing a hardware configuration example of a console unit.

FIG. 4 is an explanatory diagram showing an outline of operations by an RIS and the modality apparatus in the present embodiment.

FIG. 5 is a diagram showing an example of a setting screen for associating an examination instruction displayed on the RIS with each imaging protocol held in the modality apparatus.

FIG. 6 is a diagram showing an example of a setting screen in a case in which the examination instruction and the imaging protocol are uniquely associated with each other.

FIG. 7 is a diagram showing an example of a screen on an RIS side in a case of creating the examination order.

FIG. 8 is a diagram showing an example of an order details screen displayed on the RIS side.

FIG. 9 is a diagram showing an example of a subject registration confirmation screen displayed on a display device of the modality apparatus that has received the examination order.

FIG. 10 is a flowchart showing an example of an operation of a system including the RIS and the modality apparatus.

FIG. 11 is a block diagram showing a functional configuration of the modality apparatus according to the present embodiment.

FIG. 12 is a flowchart showing an operation in a case of subject registration in the modality apparatus that has received the examination order.

### DESCRIPTION OF THE PREFERRED EMBODIMENTS

Hereinafter, preferred embodiments of the present invention will be described in detail with reference to the accompanying drawings. In the following description and the accompanying drawings, the same components will be designated by the same reference numerals, and duplicated descriptions will be omitted.

### Configuration Example of Medical Information System

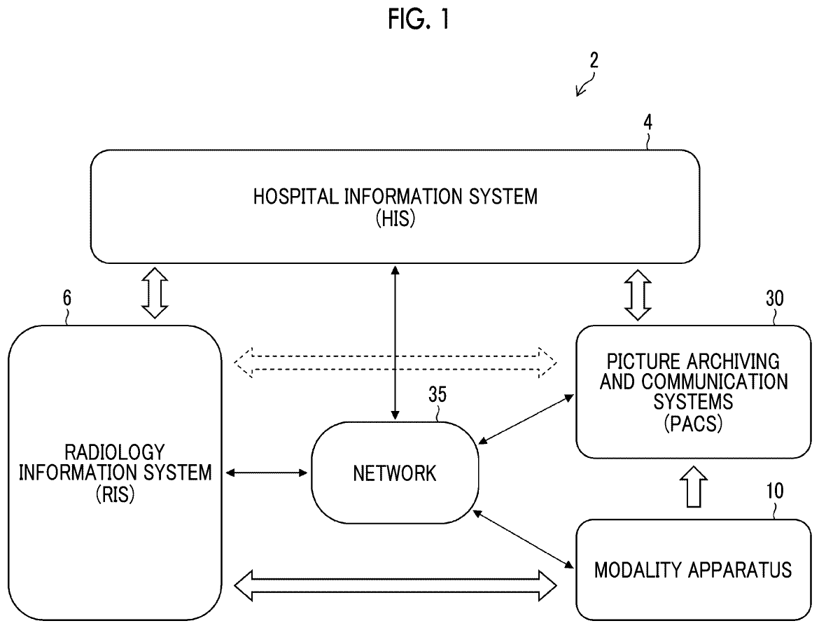

FIG. 1 is a block diagram schematically showing an exemplary configuration of a medical information system 2. The medical information system 2 is an information processing system introduced in a medical facility such as a hospital and includes a hospital information system (HIS) 4, a radiology information system (RIS) 6, a modality apparatus 10, and a picture archiving and communication systems (PACS) 30.

The HIS 4 is a system that manages various operations of the entire medical facility. The HIS 4 may include, for example, a plurality of systems such as an electronic medical record system, an ordering system, and a medical accounting system.

The RIS 6 is a system that manages operations of a radiology department (radiology division). The RIS 6 coordinates with the HIS 4 and performs, for example, processing such as examination appointment for an image diagnosis examination, creation of an examination order, and management of an examination result.

The modality apparatus 10 is an examination apparatus that captures a medical image of a subject undergoing an image diagnosis examination. A specific form of the modality apparatus 10 may be, for example, various examination apparatuses such as a magnetic resonance imaging (MRI) apparatus, a computed tomography (CT) apparatus, an ultrasound diagnostic apparatus, a positron emission tomography (PET) apparatus, a mammography apparatus, an X-ray diagnostic apparatus, an X-ray fluoroscopy diagnostic apparatus, and an endoscopic apparatus. Although FIG. 1 representatively shows one modality apparatus 10, the medical information system 2 may include a plurality of the modality apparatuses 10. There may be various combinations of types and the numbers of the modality apparatuses 10 in each medical facility.

The PACS 30 is a computer system that stores and manages various kinds of data including examination images obtained by image diagnosis examinations using the modality apparatus 10. The PACS 30 comprises a large-capacity external storage device and a database management program. The PACS 30 may include, for example, a digital imaging and communications in medicine (DICOM) server that operates according to DICOM specifications.

The HIS 4, the RIS 6, the modality apparatus 10, and the PACS 30 are communicably connected to each other via a network 35. The network 35 may be, for example, a local area network in the medical facility. A part of the network 35 may be a wide area network.

The PACS 30 receives various kinds of data including images generated by the modality apparatus 10 via the network 35, and stores and manages the data in a recording medium such as the large-capacity external storage device. The PACS 30 communicates with other devices such as the HIS 4 and the RIS 6 via the network 35 to transmit and receive various kinds of data including the examination images. The storage format of the image data in the PACS 30 and the communication between the respective devices via the network 35 are based on, for example, a DICOM protocol.

### Example of Modality Apparatus 10: MRI Apparatus

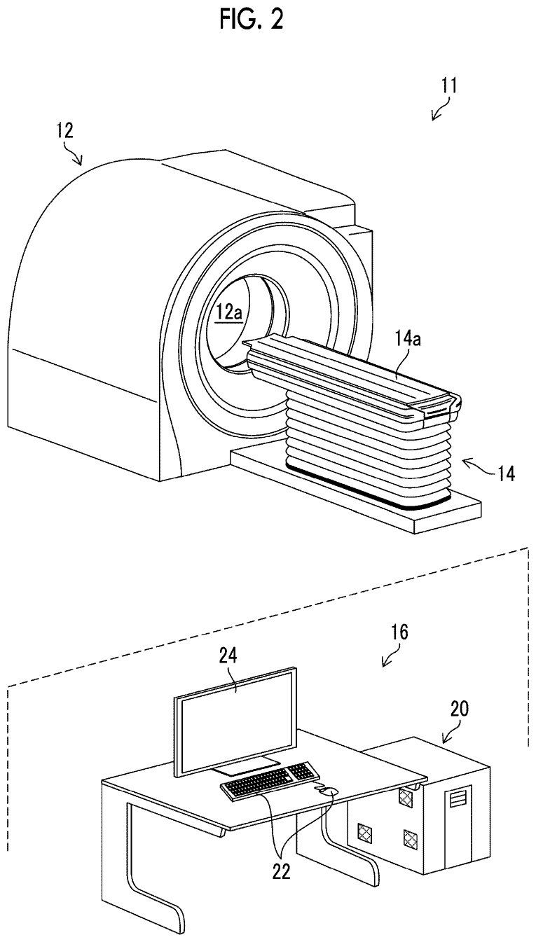

FIG. 2 is a perspective view schematically showing a configuration of an MRI apparatus 11 that is an example of the modality apparatus 10. The MRI apparatus 11 comprises a measurement unit 12 including a gantry, a patient table 14, and a console unit 16. The measurement unit 12 and the patient table 14 are disposed in an imaging room, and the console unit 16 is disposed in an operation room. In addition, although not shown, the MRI apparatus 11 comprises a gradient magnetic field power supply unit, a radio frequency (RF) amplifier unit, a helium compressor unit, a control unit, and the like. These units (not shown) are stored in a housing either individually or in a group of a plurality of units, and are disposed in a machinery room. Some or all of these units may be disposed in the operation room.

Although a detailed configuration of the measurement unit 12 is not shown, the measurement unit 12 comprises a static magnetic field generation magnet, a gradient magnetic field coil, an RF irradiation coil, an RF receive coil, and the like. The static magnetic field generation magnet is, for example, a superconducting magnet and generates a static magnetic field in an imaging space 12a of the gantry. The gradient magnetic field coil imparts a magnetic field gradient with respect to the static magnetic field generated by the static magnetic field generation magnet. The RF irradiation coil generates a high-frequency magnetic field with respect to a measurement region of the subject. The RF receive coil receives a nuclear magnetic resonance (NMR) signal generated from the measurement region of the subject.

Since the detailed configuration of each unit of the measurement unit 12 and the operation principle of the MRI apparatus 11 are well-known technologies, detailed descriptions thereof will be omitted here.

The patient table 14 comprises a tabletop 14a on which the subject undergoing the image diagnosis examination is placed. The tabletop 14a is movable in an up-down direction, in a direction of entry (forward direction) into the imaging space 12a of the gantry, and in a direction of exit (backward direction) from the imaging space 12a by a drive device (not shown).

The console unit 16 includes an information processing device 20, an input device 22, and a display device 24. The console unit 16 functions as a control apparatus that controls a control unit of the machinery room to control each unit of the measurement unit 12 and the patient table 14 and that executes MRI imaging for acquiring the NMR signal. In addition, the console unit 16 functions as an apparatus that performs processing of various kinds of data, including processing of performing image reconstruction based on the NMR signal acquired from the measurement unit 12 and communication processing through the network 35, display of a processing result, storage of data, and the like.

The NMR signal obtained from the measurement unit 12 is subjected to signal processing and then is subjected to digital image processing, and the reconstructed image is displayed on the display device 24 of the console unit 16.

### Hardware Configuration Example of Console Unit 16

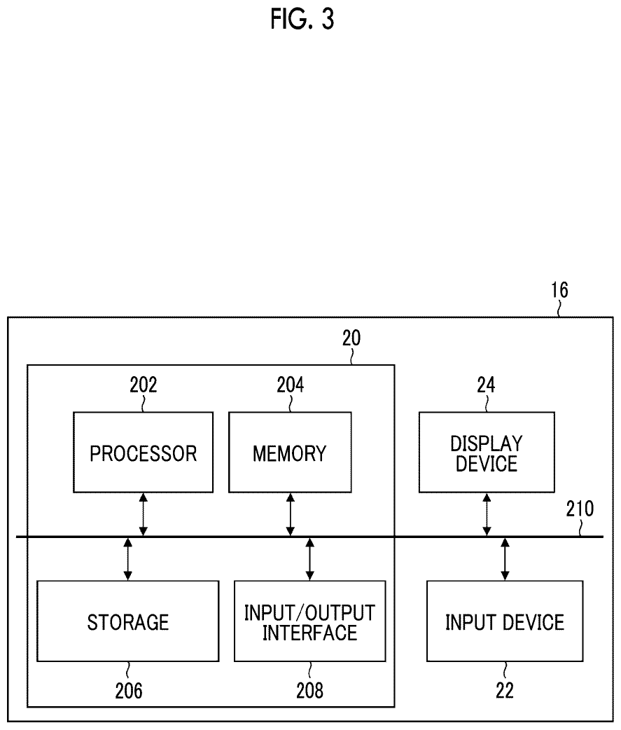

FIG. 3 is a block diagram showing a hardware configuration example of the console unit 16. The console unit 16 is configured by using a computer. The computer applied to the console unit 16 may be a personal computer, a workstation, or a server computer. The processing function of the console unit 16 may be implemented by a computer system including a plurality of computers.

The information processing device 20 of the console unit 16 comprises a processor 202, a memory 204 that is a main storage device, a storage 206 that is an auxiliary storage device, an input/output interface 208, and a bus 210.

The processor 202 includes a central processing unit (CPU). The processor 202 may include a graphics processing unit (GPU). In addition, the information processing device 20 may further include a processor such as a digital signal processor (DSP), an application specific integrated circuit (ASIC), and a programmable logic device (PLD).

The processor 202 is connected to the memory 204, the storage 206, the input/output interface 208, the input device 22, and the display device 24 via the bus 210.

The memory 204 includes a random access memory (RAM). The memory 204 may include a read only memory (ROM). The storage 206 may be, for example, a hard disk drive (HDD), a solid state drive (SSD), or a combination of a plurality of these. In addition, the storage 206 may include an external storage device such as a removable disk.

The storage device including the memory 204 and the storage 206 stores programs, data, and the like for implementing various functions of the MRI apparatus 11. The processor 202 implements various functions by executing the programs stored in the memory 204. The processor 202 comprehensively controls each unit of the information processing device 20, various devices and units provided in the MRI apparatus 11, and the like to perform various kinds of processing.

The input/output interface 208 includes a communication interface that is connectable to the network 35, a connection interface that is connectable to an external device, and the like. As the connection interface that is connectable to the external device, for example, a universal serial bus (USB), a high-definition multimedia interface (HDMI) (HDMI is a registered trademark), or the like can be applied.

The processor 202 communicates with various devices of the MRI apparatus 11 via the input/output interface 208 to transmit and receive necessary information.

The input device 22 is configured with, for example, a keyboard, a pointing device such as a mouse, a numeric keypad, various switch buttons, and the like. The input device 22 may include a voice input device. In addition, the input device 22 may be a touch panel type input device that is integrated with a display screen of the display device 24.

Various instructions or information input by the operator via the input device 22 is input to the information processing device 20. The operator interactively operates the MRI apparatus 11 by using the input device 22 and the display device 24.

The display device 24 is configured with, for example, a liquid crystal display, an organic electro-luminescence (OEL) display, a projector, or an appropriate combination thereof. The display device 24 is connected to the information processing device 20 via the bus 210. The display device 24 displays various kinds of information in addition to the examination image captured by the MRI apparatus 11. The display device 24 is used as a part of a user interface (UI) in a case of accepting an input from the input device 22. The present disclosure is not limited to one display device 24, and a form of a multi-display comprising a plurality of display devices can be used.

### Outline of Mechanism for Supporting Confirmation and Selection of Imaging Protocol Corresponding to Examination Order

A mechanism for supporting confirmation and selection of an imaging protocol corresponding to the examination order is mounted in the medical information system 2 including the modality apparatus 10 according to the present embodiment. In the present embodiment, an example will be described in which the examination order is generated by the RIS 6 and the examination order is transmitted from the RIS 6 to the modality apparatus 10, but the present disclosure is not limited to the RIS 6, and for example, a configuration may be employed in which the examination order is generated by another information system, such as an ordering system of the HIS 4, and the examination order is transmitted to the modality apparatus 10. The RIS 6 is an example of an information system.

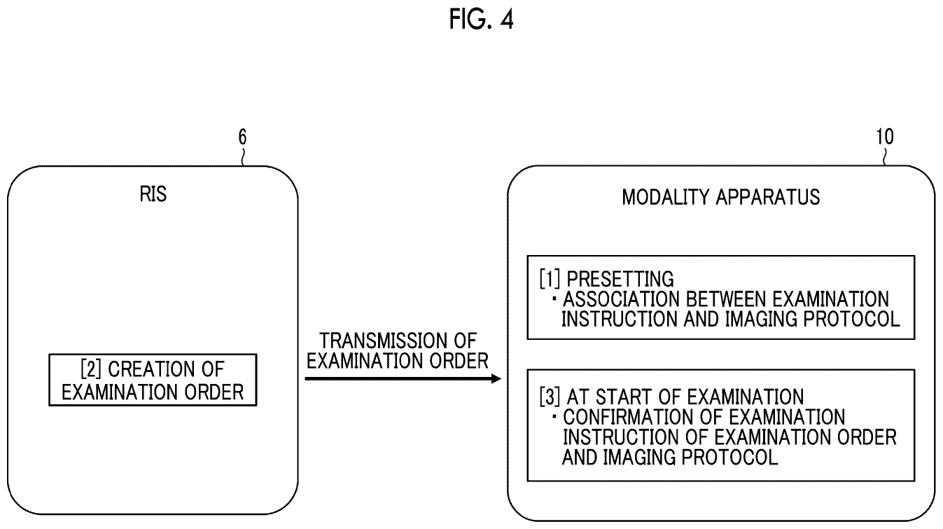

FIG. 4 is an explanatory diagram showing an outline of operations by the RIS 6 and the modality apparatus 10 in the present embodiment. A user such as a physician and a radiologist basically proceeds with their operations in the following sequence ([1]→[2]→[3]).

### [1] Presetting

First, on a modality apparatus 10 side, a setting is performed to pre-associate the examination instruction with one or more imaging protocols that are desired to be selected for the examination instruction, before the examination order is accepted from the RIS 6. The setting of this association is referred to as “presetting”. The term “association” may be rephrased as “relation” or “linkage”. The presetting is basically performed by the user and an application staff at the start of apparatus operation.

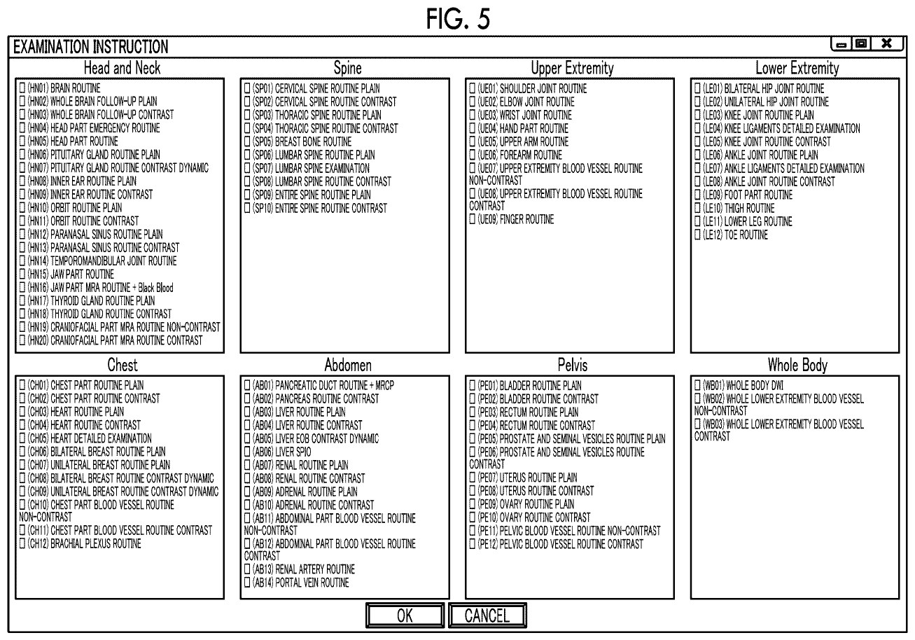

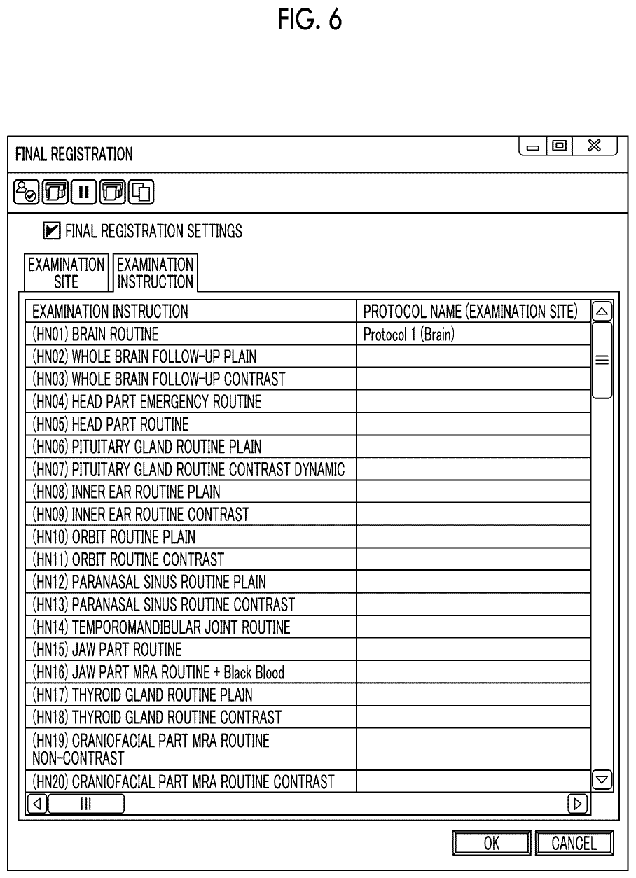

A setting screen for accepting a setting operation for the presetting by the operator is displayed on the display device 24 of the modality apparatus 10 (refer to FIGS. 5 and 6). The operator inputs necessary information through the setting screen and performs settings for associating the examination instruction and the imaging protocol with each other. Through the presetting, association information indicating a correspondence relationship between the examination instruction and the desired imaging protocol is stored in the storage device in the modality apparatus 10.

The association information set through the presetting may be updated by the operator as necessary after the initial setting. For example, there may be a case in which an imaging protocol used in clinical practice is changed or a case in which there is a desire to change the imaging protocol that is desirable to be selected for the examination instruction. Therefore, in order to respond such a need, the modality apparatus 10 has a configuration in which the association can be changed at any time on the setting screen before the start of the examination. The modality apparatus 10 accepts an operation for changing (correcting) set contents of the presetting.

In addition, by sharing the association information registered through the presetting with the RIS 6, it is possible to create the examination order by using the association information on an RIS 6 side.

### [2] Creation of Examination Order

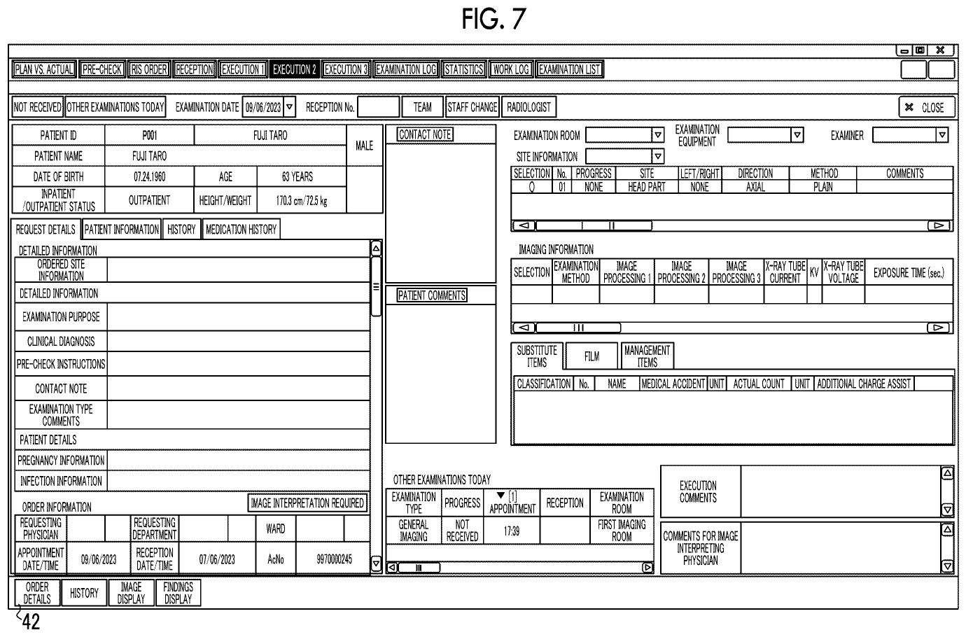

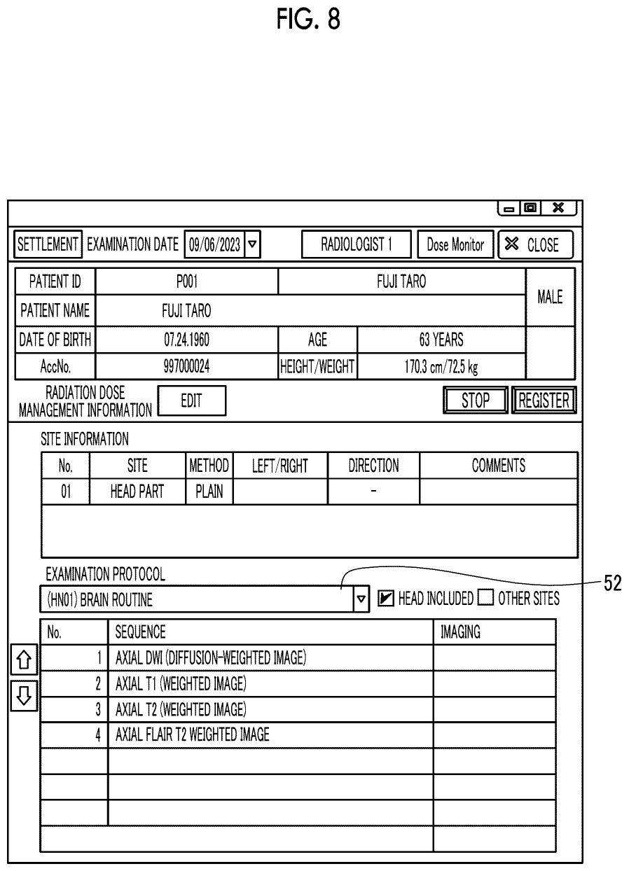

In a case in which the image diagnosis examination is performed, the examination order is created on the RIS 6 side. A physician who requests the examination inputs necessary information for the examination, such as subject information, an examination purpose, an examination site, an examination instruction, and an imaging protocol, by using a screen of a terminal of the RIS 6, and creates the examination order (refer to FIGS. 7 and 8). In a case of creating this examination order, the association information of the presetting can be utilized. An imaging protocol suitable for the examination instruction can be designated in accordance with the association information. The created examination order is transmitted from the RIS 6 to the modality apparatus 10.

### [3] Confirmation of Examination Instruction of Examination Order and Imaging Protocol on Modality Apparatus 10 Side at Start of Examination

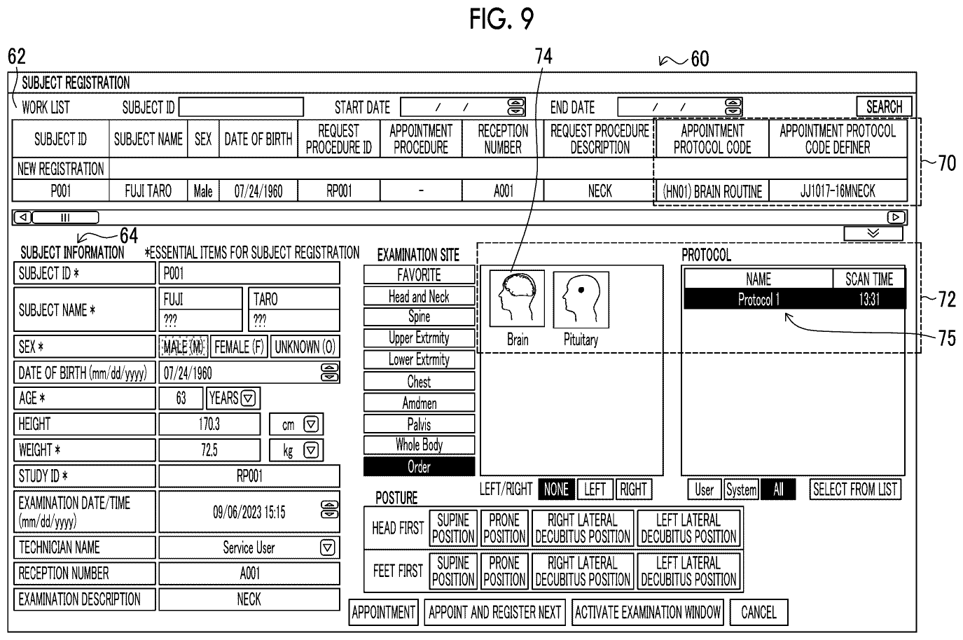

On the modality apparatus 10 side that has received the examination order, information including the examination instruction designated in the examination order and the imaging protocol associated with the examination instruction is displayed on the display device 24. As a result, the operator such as the radiologist who operates the modality apparatus 10 can confirm, on the screen of the display device 24, that the examination order designated by the physician and the imaging protocol to be selected are correct (refer to FIG. 9).

In a case in which one specific imaging protocol is uniquely associated with the examination instruction designated in the received examination order through the presetting, the modality apparatus 10 may automatically select an appropriate imaging protocol for the examination instruction of the examination order from the preset association information. In addition, in a case in which a plurality of imaging protocols are associated with the examination instruction, the modality apparatus 10 can display, on the display device 24, selection candidates of the plurality of imaging protocols associated with the examination instruction from the preset association information, and display a screen for prompting the operator to select an appropriate imaging protocol from among the selection candidates. The operator selects the imaging protocol from among the displayed selection candidates, thereby determining the imaging protocol to be applied to the examination.

[2] and [3] described above are performed for each examination every time. [1] is performed at the initial setting or in a case in which subsequent setting changes are required. Hereinafter, further specific examples will be described.

### Regarding Internal Processing Related to Presetting

A specific example of a mechanism for coordination using identification information in a case of coordinating the RIS 6 and the modality apparatus 10 for information regarding the examination order will be described.

The RIS 6 and the modality apparatus 10 generally communicate with each other in accordance with the DICOM standard. Therefore, in order for the RIS 6 and the modality apparatus 10 to recognize information contents in common, how to use a DICOM tag for coordination is determined in advance. The use of the DICOM standard is not essential, but an example of using the DICOM standard will be described here.

In the present embodiment, an example will be described in which a tag A and a tag B in the DICOM tag are used for the coordination between the RIS 6 and the modality apparatus 10.

A common symbol is added to the tag A. The common symbol is an unchangeable symbol used in common for the RIS 6 and the modality apparatus 10. In the modality apparatus 10, through the presetting, the common symbol is associated with the imaging protocol held in the modality apparatus 10. For the common symbol, a specific symbol is defined for each type of the examination instruction. For example, a common symbol “HN01” is defined for an examination instruction called “brain routine”. The common symbol is an example of identification information and functions as identification (ID). In the DICOM standard, the tag A is not determined as a tag including a common symbol associated with the imaging protocol, but is determined uniquely in the present embodiment.

Information (for example, a keyword character string) indicating that it is a coordination target is added to the tag B. In a case in which the value of the tag B matches the specific keyword character string, processing of using the value (common symbol) of the tag A as a key to select an imaging protocol associated with the key in the modality apparatus 10 is executed. The information added to the tag B is an example of coordination necessity information indicating the necessity of coordination. In the DICOM standard, the tag B is not determined as a tag for coordination, but is determined uniquely in the present embodiment.

### Example of Graphical User Interface (GUI) in Presetting

FIG. 5 shows an example of a screen for associating the examination instruction displayed on the RIS 6 with each imaging protocol held in the modality apparatus 10.

Inside the modality apparatus 10, a correspondence table of an RIS protocol ID defined for each examination instruction displayed on the RIS 6 is stored, and the RIS protocol ID and the imaging protocol are associated with each other through the presetting. The RIS protocol ID is the common symbol used in common in the RIS 6 and the modality apparatus 10.

A list of the selection candidates of the examination instruction shown in FIG. 5 is composed of notations of the names of the examination instructions displayed on the RIS 6, and the symbol in parentheses in a parenthesized part, such as “(HN01)”, in the name of the examination instruction represents the RIS protocol ID defined for the examination instruction. For example, an RIS protocol ID “HN01” is defined for “(HN01) brain routine”, which is the examination instruction displayed on the RIS 6. In addition, an RIS protocol ID “HN02” is defined for “(HN02) whole brain follow-up plain”, which is the examination instruction displayed on the RIS 6. The RIS protocol ID corresponds to the common symbol described above. The name (notation) of the examination instruction displayed on the RIS 6 can be freely determined on the RIS 6 side.

As shown in FIG. 5, the examination instruction is classified for each category of the examination site. For the imaging protocol registered in the modality apparatus 10, a plurality of candidates of examination instructions to which the imaging protocol is applied can be selected for each category of the corresponding examination site. The selection candidate of the examination instruction can be set for each imaging protocol held in the modality apparatus 10 on a selection screen shown in FIG. 5. Through this setting, the association between the examination instruction and the imaging protocol is registered.

The presetting includes provisional registration and final registration. The provisional registration is a setting for associating the RIS protocol ID with a plurality of imaging protocols related to the examination site. That is, the setting for associating the plurality of imaging protocols with the RIS protocol ID is referred to as “provisional registration”. The final registration is a setting for uniquely associating one imaging protocol with the RIS protocol ID.

FIG. 6 is an example of a setting screen in a case in which the examination instruction and the imaging protocol are uniquely associated with each other (final registration). FIG. 6 shows an example in which an imaging protocol with a protocol name “Protocol1” is associated with an examination instruction “(HN01) brain routine” shown in the first line of the list. A parenthesized part “(Brain)” of the notation “Protocol1 (Brain)” in FIG. 6 indicates that the examination target site is the brain. In addition, a specific sequence and condition are defined in the imaging protocol with the protocol name “Protocol1”. A specific imaging protocol can be set uniquely for each of the other examination instructions shown in the second and subsequent lines of FIG. 6. A pair of the examination instruction and the imaging protocol associated through the final registration can be identified by the RIS protocol ID, which is the common symbol between the RIS 6 and the modality apparatus 10. In order to identify a pair of the examination instruction and the imaging protocol, a common symbol (identification symbol) that can be recognized in common by the RIS 6 and the modality apparatus 10 may be added to the pair.

### Screen Example on RIS 6 Side in Case of Creating Examination Order

FIG. 7 is an example of a screen on the RIS side in a case of creating the examination order. On the screen of the RIS shown in FIG. 7, the physician inputs necessary information for the examination. The RIS 6 may automatically acquire information such as subject information from another system in coordination with an electronic medical record system or the like. On the screen of FIG. 7, for example, in a case in which an order details button 42 is pressed, an order details screen as shown in FIG. 8 is displayed.

On the order details screen shown in FIG. 8, the physician designates the examination site and the imaging protocol that match the examination purpose. In a combo box 52 for accepting an input of the examination instruction, the physician can select the examination instruction that matches the examination purpose. The name of the examination instruction displayed in a drop-down list of the combo box 52 is “the examination instruction displayed on the RIS 6”. In a case of coordinating information regarding the imaging protocol between the RIS 6 and the modality apparatus 10, for example, by selecting a preset examination instruction, such as “(HN01) brain routine”, an examination order including information on the imaging protocol associated with the examination instruction can be created. The examination order created by using the preset association information includes the keyword character string (an example of coordination necessity information) indicating that it is a coordination target using the association information, and the RIS protocol ID (an example of a common symbol) used in common in the RIS 6 and the modality apparatus 10.

The combo box 52 of the examination instruction allows not only an input of a pre-registered examination instruction but also an input of an examination instruction in a free-form format, such as text input, for example.

### Screen Example of Confirmation and Selection on GUI on Modality Apparatus 10 Side at Start of Examination

FIG. 9 is a diagram showing an example of a subject registration confirmation screen 60 displayed on the display device 24 of the modality apparatus 10 that has received the examination order. The subject registration confirmation screen 60 includes a work list display area 62 and a registration information display area 64.

In the work list display area 62, a work list corresponding to the examination order received from the RIS 6 is displayed. In FIG. 9, only the examination order for a newly registered subject (subject ID: P001) is shown, but a list of examination orders of a plurality of subjects may be displayed in the work list display area 62.

The work list display area 62 includes a column for displaying an appointment protocol code and a column for displaying an appointment protocol code definer as in a part surrounded by a dashed line frame 70. The appointment protocol code corresponds to the RIS protocol ID of the examination instruction designated in the examination order. In the column for displaying the appointment protocol code, the name of the examination instruction corresponding to the RIS protocol ID of the examination instruction designated in the examination order is displayed. The processor 202 converts the received RIS protocol ID into the name of the examination instruction displayed on the RIS 6 and displays the name of the examination instruction in the appointment protocol code field.

The appointment protocol code definer corresponds to information (keyword character string) indicating that it is the coordination target. In a case in which the received examination order is the coordination target, the keyword character string of the tag A is displayed in the column for displaying the appointment protocol code definer. It is sufficient to indicate that it is the coordination target in a case in which the appointment protocol code definer is displayed, and that it is not the coordination target in a case in which the appointment protocol code definer is not displayed. There is no intention to prompt the user to confirm the correctness of information on the appointment protocol code definer.

The registration information display area 64 displays detailed registration information regarding the examination order of the subject selected in the work list. The registration information display area 64 includes a subject information display area, an examination site display area, and a protocol display area. In the subject information display area, information for specifying the subject and information regarding an attribute of the subject are displayed. In the examination site display area, information regarding the examination site designated in the examination order is displayed. In the protocol display area, the imaging protocol corresponding to the examination instruction designated in the examination order is displayed.

As in a part surrounded by a dashed line frame 72 in FIG. 9, on the subject registration confirmation screen 60, an icon 74 indicating the examination site of the imaging protocol corresponding to the examination instruction and an imaging protocol 75 are displayed with the RIS protocol ID designated in the examination order as a key, based on the set contents of the presetting.

In a case in which the final registration for the RIS protocol ID designated in the examination order has been completed, the selection of the finally registered imaging protocol 75 is automatically executed. In a case in which the final registration for the RIS protocol ID designated in the examination order has not been made, and only the provisional registration has been made, the first icon is selected in the listed icons 74 indicating the examination site, and the imaging protocol is not automatically selected. In this case, the protocol display area displays the selection candidates of a plurality of imaging protocols set on the provisional registration setting screen (refer to FIG. 5), and the operator is prompted to perform a selection operation for designating the imaging protocol.

### Operations by RIS 6 and Modality Apparatus 10

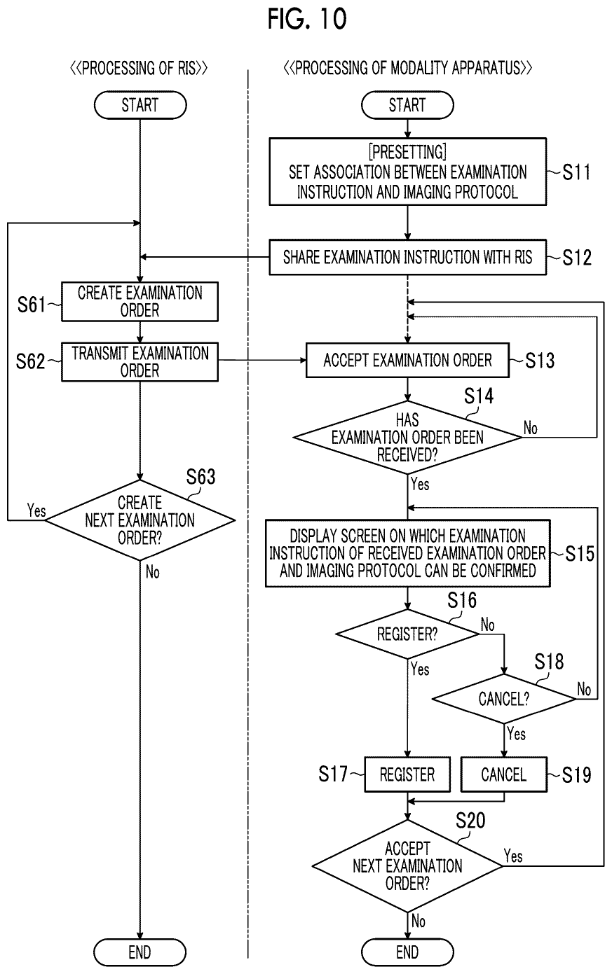

FIG. 10 is a flowchart showing an example of the operation of the system including the RIS 6 and the modality apparatus 10. A flowchart shown in FIG. 10 is an example of an information processing method in the present disclosure.

The right side of FIG. 10 shows steps (steps S11 to S20) of processing of the modality apparatus 10, and the left side of FIG. 10 shows steps (steps S62 and S63) of processing of the RIS 6.

In step S11, the processor 202 of the modality apparatus 10 accepts the presetting operation and performs the setting for pre-associating the examination instruction and the imaging protocol in accordance with the setting operation by the operator. The association information set through the presetting includes the provisionally registered provisional registration data and the finally registered final registration data.

In step S12, the processor 202 of the modality apparatus 10 may share the examination instruction corresponding to the presetting with the RIS 6. After that, the processor 202 accepts the examination order from the RIS 6 (step S13).

Meanwhile, in step S61, the RIS 6 creates the examination order based on an operation of the physician or the like who requests the examination. In a case of creating the examination order, by using examination instruction information, the RIS 6 and the modality apparatus 10 can coordinate with each other.

In step S62, the RIS 6 transmits the created examination order to the modality apparatus 10. After that, in step S63, the RIS 6 determines whether or not to create the next examination order. In step S63, in a case in which the RIS 6 determines to continue creation processing of the next examination order (in a case in which a determination result of step S63 is Yes), the RIS 6 returns to step S61. On the other hand, in step S63, in a case in which the RIS 6 determines to end the creation processing of the examination order (in a case in which the determination result of step S63 is No), the RIS 6 ends the processing shown in the flowchart of FIG. 10.

After starting to accept the examination order in step S13, the processor 202 of the modality apparatus 10 determines in step S14 whether or not the examination order has been received. In a case in which the modality apparatus 10 has not received the examination order from the RIS 6, that is, in a case in which the determination result in step S14 is No, the processor 202 returns to step S13 and waits for the reception of the examination order from the RIS 6.

On the other hand, in a case in which the modality apparatus 10 has received the examination order from the RIS 6 (in a case in which the determination result in step S14 is Yes), the processor 202 transitions to step S15.

In step S15, the processor 202 displays a screen (refer to FIG. 9) on which the operator can confirm the examination instruction of the received examination order and the corresponding imaging protocol. With this screen display, the operator can confirm whether or not the imaging protocol corresponding to the examination instruction designated by the physician is appropriate. The processor 202 accepts operations such as changing or selecting the information displayed on the subject registration confirmation screen 60. The operator confirms in step S15 whether or not there is an error in the displayed content, and in a case in which the displayed content needs to be changed, selected, or the like, the operator can perform operations such as changes and selections on this subject registration confirmation screen 60.

In step S16, the processor 202 determines whether or not to register the execution or the appointment of the examination with the examination contents displayed on the subject registration confirmation screen 60. In a case in which the operator confirms the screen display to perform an operation of registering the execution or the appointment of the examination, the determination result in step S16 is Yes, and the process transitions to step S17.

In step S17, the processor 202 executes subject registration for the examination based on the content of information displayed on the subject registration confirmation screen 60.

On the other hand, in a case in which the determination result in step S16 is No, the processor 202 transitions to step S18. In step S18, the processor 202 determines whether or not to cancel the display or the operations such as the changes or the selections accepted on the subject registration confirmation screen 60. In a case in which the operator does not press a cancel button on the subject registration confirmation screen 60, the determination result in step S18 is No, and the process returns to step S15. In a case in which the cancel button is pressed on the subject registration confirmation screen 60, the determination result in step S18 is Yes, and the process transitions to step S19.

In step S19, the processor 202 cancels the operation and the like accepted on the subject registration confirmation screen 60 and transitions to step S20. In step S20, the processor 202 determines whether or not to accept the next examination order.

In step S20, in a case in which the processor 202 determines to continue processing of accepting the next examination order (in a case in which the determination result in step S20 is Yes), the processor 202 returns to step S13. On the other hand, in step S20, in a case in which the processor 202 determines to end the processing of accepting the examination order (in a case in which the determination result in step S20 is No), the processor 202 ends the processing shown in the flowchart of FIG. 10.

### Functional Configuration of Modality Apparatus 10

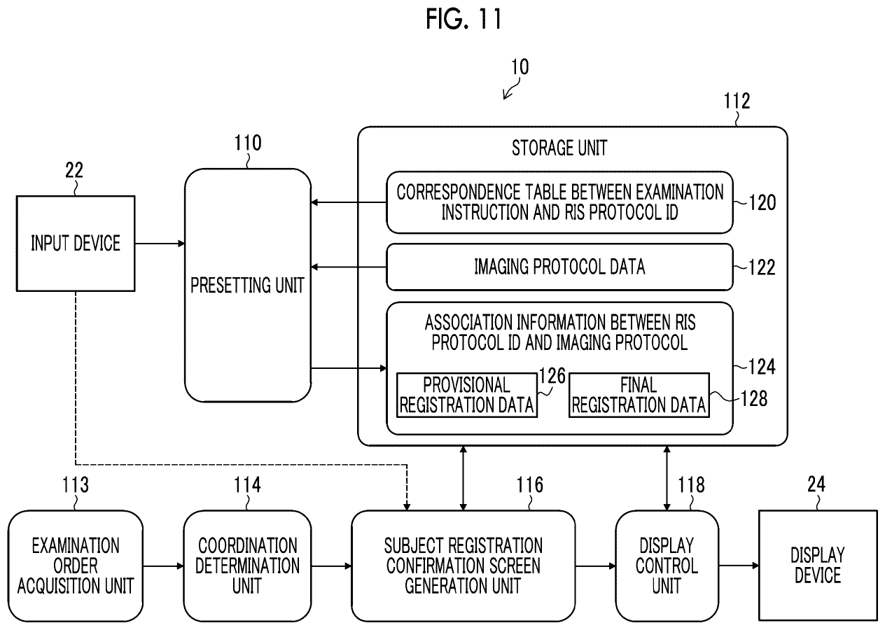

FIG. 11 is a block diagram showing a functional configuration of the modality apparatus 10 according to the present embodiment. The modality apparatus 10 comprises a presetting unit 110, a storage unit 112, an examination order acquisition unit 113, a coordination determination unit 114, a subject registration confirmation screen generation unit 116, and a display control unit 118.

The presetting unit 110 accepts the presetting operation via the input device 22 and performs processing for the presetting of associating the examination instruction and the imaging protocol.

The storage unit 112 stores a correspondence table 120 between the examination instruction displayed on the RIS 6 and the RIS protocol ID, and imaging protocol data 122. The imaging protocol data includes data of a plurality of imaging protocols. In addition, the storage unit 112 stores association information 124 between the RIS protocol ID and the imaging protocol set by the presetting unit 110. The association information 124 includes provisional registration data 126 and final registration data 128. The storage unit 112 may be a storage device including the memory 204 and the storage 206 shown in FIG. 3.

The examination order acquisition unit 113 acquires the examination order from the RIS 6. The coordination determination unit 114 determines the necessity of coordination based on the information on the tag A and the tag B of the acquired examination order.

The subject registration confirmation screen generation unit 116 generates the subject registration confirmation screen 60 based on the acquired examination order. In a case in which the acquired examination order is the coordination target, the subject registration confirmation screen generation unit 116 generates the subject registration confirmation screen 60 including the display of the imaging protocol corresponding to the examination instruction of the examination order by using the association information 124 between the RIS protocol ID and the imaging protocol held in the storage unit 112.

The display control unit 118 generates a display signal necessary for display output to the display device 24 and performs display control of the display device 24. The display control unit 118 displays the subject registration confirmation screen 60 generated by the subject registration confirmation screen generation unit 116 on the display device 24.

### Regarding Hardware Configuration of Each Processing Unit

The hardware structure of the processing unit that executes various kinds of processing, such as the presetting unit 110, the examination order acquisition unit 113, the coordination determination unit 114, the subject registration confirmation screen generation unit 116, and the display control unit 118 in the modality apparatus 10 shown in FIG. 11 is, for example, various processors to be described below.

The various processors include a CPU, which is a general-purpose processor that executes programs to function as various processing units, a GPU, a programmable logic device (PLD), which is a processor whose circuit configuration is changeable after manufacturing, such as a field programmable gate array (FPGA), a dedicated electrical circuit, which is a processor having a dedicated circuit configuration designed to execute specific processing, such as an application specific integrated circuit (ASIC), and the like.

One processing unit may be configured with one of the various processors or may be configured with two or more processors of the same type or different types. For example, one processing unit may be configured with a plurality of FPGAs, a combination of a CPU and an FPGA, or a combination of a CPU and a GPU. Additionally, a plurality of processing units may be configured with one processor. An example of configuring a plurality of processing units with one processor includes, firstly, an aspect of configuring one processor by combining one or more CPUs and software, in which the processor functions as the plurality of processing units, as typified by a computer such as a client and a server. Secondly, there is an aspect of using a processor that implements the functions of the entire system including a plurality of processing units with one integrated circuit (IC) chip, as typified by a system on chip (SoC). As described above, various processing units are configured by using one or more of the above various processors as the hardware structure.

Further, the hardware structure of these various processors is, more specifically, an electrical circuit (circuitry) in which circuit elements such as semiconductor elements are combined.

### Processing Flow of Subject Registration in Modality Apparatus 10

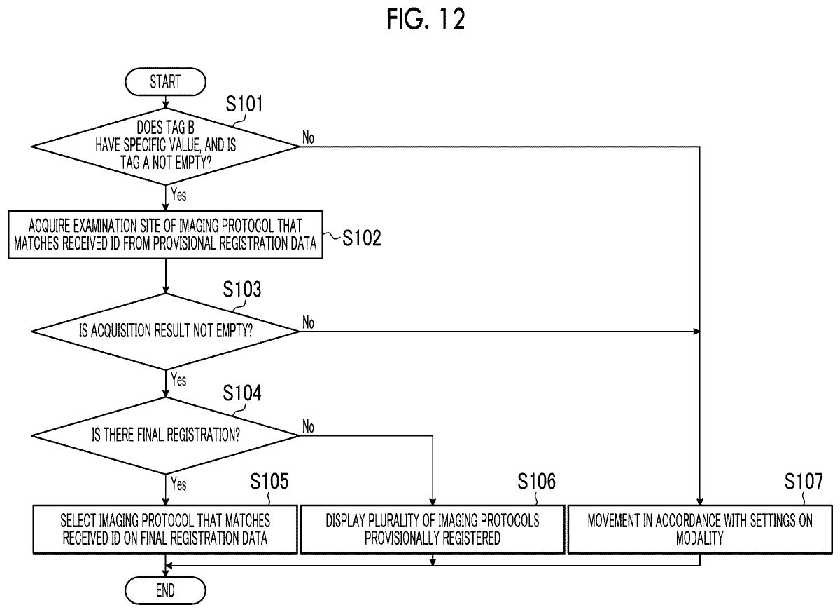

FIG. 12 is a flowchart showing an operation in a case of the subject registration in the modality apparatus 10 that has received the examination order.

In step S101, the processor 202 determines whether the tag B has a specific value and the tag A is not empty. This determination processing is processing of determining whether to check the provisional registration data 126 using the provisional registration function. In a case in which the tag B has the specific value and the tag A is not empty, the processor 202 transitions to step S102.

In step S102, the processor 202 acquires the examination site of the imaging protocol that matches the received RIS protocol ID, from the provisional registration data 126.

In step S103, the processor 202 determines whether the acquisition result in step S102 is not empty. The determination processing in step S103 is processing of determining whether there is a provisional registration for the received RIS protocol ID.

In a case in which the acquisition result is not empty as a result of the determination in step S103, the processor 202 transitions to step S104. In step S104, the processor 202 determines whether there is a final registration for the received RIS protocol ID.

In a case in which it is determined that there is a final registration as a result of the determination in step S104, the processor 202 transitions to step S105. In step S105, the processor 202 selects the imaging protocol that matches the received RIS protocol ID in accordance with the final registration data 128.

On the other hand, in a case in which it is determined that there is no final registration as a result of the determination in step S104, the processor 202 transitions to step S106.

In step S106, the processor 202 displays the plurality of imaging protocols provisionally registered for the received RIS protocol ID. In the screen display, the processor 202 accepts the selection of the imaging protocol from the operator.

On the other hand, in a case in which the tag B has no specific value, or in a case in which the tag A is empty, as a result of the determination in step S101, this means that the conventional method of receiving the examination order is used. In this case, the processor 202 transitions to step S107. In addition, in a case in which the acquisition result is empty as a result of the determination in step S103, the processor 202 transitions to step S107. In step S107, the processor 202 operates in accordance with the settings on the modality apparatus side based on the conventional function. The conventional function indicates a function of a conventional modality apparatus that does not use a mechanism of the coordination based on the RIS protocol ID implemented in the present embodiment. Specifically, for example, the favorite section is displayed or the manual input screen is displayed, thereby allowing the operator to select the imaging protocol by himself/herself.

After step S105, step S106, or step S107, the flowchart of FIG. 12 ends. The flowchart of FIG. 12 may be performed for each received examination order.

### Effects of Embodiment

With the medical information system 2 including the modality apparatus 10 and the RIS 6 according to the present embodiment, the following effects can be obtained.

[1] The modality apparatus 10 can display the imaging protocol associated with the examination instruction for the examination order received from the RIS 6 at the start of the examination, based on the preset association information. As a result, the operator of the modality apparatus 10 can easily confirm the imaging protocol corresponding to the examination order designated by the physician and can select an appropriate imaging protocol that is suitable for the examination order.

[2] According to the present embodiment, the workload of the operator of the modality apparatus 10 is reduced, and selection errors of the imaging protocol can be suppressed. In addition, since the imaging protocol suitable for the examination order can be selected based on the preset association information, the examination can be started promptly after the examination order is received, thereby avoiding a situation in which the patient has to wait.

[3] Since the set contents of the presetting can be changed at any time, it is possible to flexibly respond to a change in the content of the imaging protocol after the start of the system operation or a change in a clinical situation.

[4] By making it possible to create the examination order by utilizing the preset association information, the burden on the physician who creates the examination order is reduced.

### Regarding Program for Operating Computer

A program for causing a computer to implement some or all of the processing functions of the information processing device 20 in the modality apparatus 10 according to the present embodiment is recorded on a computer-readable medium, which is a tangible non-transitory information storage medium, such as an optical disk, a magnetic disk, or a semiconductor memory, and the program can be provided through the information storage medium.

In addition, instead of the aspect in which the program is stored in the tangible non-transitory computer-readable medium and provided, a program signal can also be provided as a download service by using an electrical communication line such as the Internet.

Further, some or all of the processing functions in the information processing device 20 may be implemented by cloud computing, and the information processing device 20 can also be provided as software as a service (SaaS).

### Others

The present disclosure is not limited to the above embodiments, and various modifications can be made within the scope that does not departing from the gist of the technical idea of the present disclosure.

### EXPLANATION OF REFERENCES

2: medical information system
4: hospital information system (HIS)
6: radiology information system (RIS)
10: modality apparatus
11: MRI apparatus
12: measurement unit
12a: imaging space
14: patient table
14a: tabletop
16: console unit
20: information processing device
22: input device
24: display device
30: picture archiving and communication systems (PACS)
35: network
42: order details button
52: combo box
60: subject registration confirmation screen
62: work list display area
64: registration information display area
70, 72: dashed line frame
74: icon
75: imaging protocol
110: presetting unit
112: storage unit
113: examination order acquisition unit
114: coordination determination unit
116: subject registration confirmation screen generation unit
118: display control unit
120: correspondence table between examination instruction displayed on RIS and RIS protocol ID
122: imaging protocol data
124: association information between RIS protocol ID and imaging protocol
126: provisional registration data
128: final registration data
202: processor
204: memory
206: storage
208: input/output interface
210: bus
S11 to S20: steps of processing in modality apparatus
S61 to S63: steps of processing in RIS
S101 to S107: steps of processing of subject registration in modality apparatus

What is claimed is:

1. A medical information system comprising:
an information system that generates an examination order including an examination instruction for an image diagnosis examination; and
a modality apparatus that captures a medical image of a subject in accordance with the examination order,
wherein the modality apparatus includes
a storage device that pre-associates and stores the examination instruction and one or more imaging protocols, and
one or more processors that execute processing of acquiring the examination order from the information system and displaying the examination instruction of the acquired examination order and the one or more imaging protocols pre-associated with the examination instruction.

2. The medical information system according to claim 1,
wherein the one or more processors
execute processing of displaying a setting screen for accepting a setting operation to associate the one or more imaging protocols corresponding to the examination instruction.

3. The medical information system according to claim 1,
wherein the one or more processors
execute processing for provisional registration of storing identification information determined for each type of the examination instruction and a plurality of imaging protocols related to an examination site according to the examination instruction in association with each other in the storage device.

4. The medical information system according to claim 3,
wherein the one or more processors display the plurality of imaging protocols associated with the identification information and display a screen for prompting an operator to select any of the imaging protocols, in a case in which the provisional registration has been made for the examination instruction of the examination order acquired from the information system.

5. The medical information system according to claim 1,
wherein the one or more processors execute processing for final registration of storing identification information determined for each type of the examination instruction and one imaging protocol in association with each other in the storage device.

6. The medical information system according to claim 5,
wherein the one or more processors display the imaging protocol uniquely associated with the identification information and execute automatic selection of the uniquely associated imaging protocol, in a case in which the final registration has been made for the examination instruction of the examination order acquired from the information system.

7. The medical information system according to claim 1,
wherein the examination order includes coordination necessity information indicating a necessity of coordination between the information system and the modality apparatus.

8. The medical information system according to claim 7,
wherein the one or more processors control whether or not to display the pre-associated one or more imaging protocols according to the coordination necessity information included in the examination order acquired from the information system.

9. The medical information system according to claim 1,
wherein the examination order includes identification information determined for the examination instruction.

10. The medical information system according to claim 1,
wherein a common symbol that is unchangeable between the information system and the modality apparatus is assigned to a pair of the pre-associated examination instruction and imaging protocol.

11. A modality apparatus that captures a medical image of a subject in accordance with an examination order including an examination instruction for an image diagnosis examination, the modality apparatus comprising:
a storage device that pre-associates and stores the examination instruction and one or more imaging protocols; and
one or more processors that execute processing of acquiring the examination order from an information system and displaying the examination instruction of the acquired examination order and the one or more imaging protocols pre-associated with the examination instruction.

12. An information processing method of coordinating an information system that generates an examination order including an examination instruction for an image diagnosis examination with a modality apparatus that captures a medical image of a subject in accordance with the examination order, the information processing method comprising:
causing one or more processors of the modality apparatus to pre-associate and store the examination instruction and one or more imaging protocols in a storage device; and
causing the one or more processors to execute processing of acquiring the examination order from the information system and of displaying the examination instruction of the acquired examination order and the one or more imaging protocols pre-associated with the examination instruction.

13. A non-transitory, computer readable tangible recording medium which records thereon a program for causing, when read by a computer, the computer to implement the information processing method according to claim 12.
